(19) United States
(12) Patent Application Publication
(10) Pub. No.: US 2013/0144283 A1
(43) Pub. Date: Jun. 6, 2013

(54) TREATMENT OF POLYCYSTIC OVARY SYNDROME USING RENAL NEUROMODULATION AND ASSOCIATED SYSTEMS AND METHODS

(71) Applicant: Medtronic Ardian Luxembourg S.a.r.l., Luxembourg (LU)

(72) Inventor: Neil C. Barman, Menlo Park, CA (US)

(73) Assignee: Medtronic Ardian Luxembourg S.a.r.l., Luxembourg (LU)

(21) Appl. No.: 13/691,556

(22) Filed: Nov. 30, 2012

Related U.S. Application Data
(60) Provisional application No. 61/566,576, filed on Dec. 2, 2011.

(51) Int. Cl. A61B 18/02; A61B 18/04; A61B 17/00

(52) U.S. Cl. CPC A61B 18/02; A61B 17/00; A61B 18/04
USPC 606/20; 128/898; 606/27

(57) ABSTRACT

Methods for treating polycystic ovary syndrome with therapeutic renal neuromodulation and associated systems and methods are disclosed herein. The polycystic ovary syndrome can be associated, for example, with a condition including at least one of oligo/amenorrhea, infertility, hirsutism, obesity, metabolic syndrome, insulin resistance, and increased cardiovascular risk profile. One aspect of the present technology is directed to methods that at least partially inhibit sympathetic neural activity in nerves proximate a renal artery of a kidney of a patient. Central sympathetic drive in the patient can thereby be reduced in a manner that treats the patient for the polycystic ovary syndrome. Renal sympathetic nerve activity can be modulated along the afferent and/or efferent pathway. The modulation can be achieved, for example, using an intravascularly positioned catheter carrying a therapeutic assembly, e.g., a therapeutic assembly configured to cryotherapeutically cool the renal nerve or to deliver an energy field to the renal nerve.

### CROSS-REFERENCE TO RELATED APPLICATION(S)

The present application claims priority to U.S. Provisional Patent Application No. 61/566,576, filed Dec. 2, 2011, entitled “TREATMENT OF POLYCYSTIC OVARY SYNDROME USING RENAL NEUROMODULATION,” which is incorporated herein in its entirety by reference.

### TECHNICAL FIELD

The present technology relates generally to polycystic ovary syndrome and related conditions. In particular, several embodiments are directed to treatment of polycystic ovary syndrome and related conditions using renal neuromodulation and associated systems and methods.

### BACKGROUND

Polycystic ovary syndrome (PCOS) is a common endocrine disorder affecting women of reproductive ages (e.g., 12-45 years old). Symptoms of PCOS can include oligoovulation or anovulation resulting in irregular menstruation, amenorrhea, ovulation-related infertility, and enlarged or polycystic ovaries. Other symptoms include excess of androgenic hormones (e.g., testosterone) which can result in acne and hirsutism. Clinical complications, such as insulin resistance, obesity, Type 2 diabetes, high cholesterol, and hypertension can also be common in PCOS patients. Further complications can include development of endometrial cancer or breast cancer. Most prescribed treatments address specific manifestations of PCOS and do not address underlying causes of the disease. For example, androgen excess and associated symptoms (e.g., hirsutism, acne) are commonly treated with estrogen-progestin contraceptives, antiandrogens, anti-acne treatments, and prescription drugs and over-the-counter depilatories for removing or slowing unwanted hair growth. Additionally, anovulation and fertility issues are treated with ovulation promoting drugs (e.g., clomiphene or follicle stimulating hormone (FSH) injections) or in vitro fertilization. Other treatments are prescribed for PCOS patients having hypertension (e.g., anti-hypertensive medications), hyperlipidemia (e.g., statins, other cholesterol lowering agents), and insulin-resistance/Type 2 diabetes (e.g., metformin, other diabetic medications). Such pharmacologic strategies, however, have significant limitations including limited efficacy, side effects, long-term maintenance regimens and others.

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. As examples, radiotracer dilution has demonstrated increased renal norepinephrine (NE) spillover rates in patients with essential hypertension, and elevated sympathetic nervous system activity has been shown to be present in PCOS.

Sympathetic nerves of the kidneys terminate in the blood vessels, the juxtaglomerular apparatus, and the renal tubules. Stimulation of the renal sympathetic nerves can cause increased renin release, increased sodium (Na⁺) reabsorption, and a reduction of renal blood flow. These neural regulation components of renal function are considerably stimulated in disease states characterized by heightened sympathetic tone as well as likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), calcium channel blockers and vasodilators (to counteract peripheral vasoconstriction caused by increased sympathetic drive), aldosterone blockers (to block the actions of increased aldosterone released from activation of the renin-angiotensin-aldosterone system), and diuretics (intended to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a plot of systolic office blood pressure (mmHg) at a baseline assessment and at 12 weeks after renal neuromodulation for two patients with polycystic ovary syndrome (PCOS).

FIG. 1B is a plot of muscle sympathetic nerve activity (bursts per 100 heart beats) at a baseline assessment and at 12 weeks after renal neuromodulation for two patients with PCOS.

FIG. 1C is a plot of whole body norepinephrine spillover (ng/min) at a baseline assessment and at 12 weeks after renal neuromodulation for two patients with PCOS.

FIG. 2A is a plot of body weight (kg) at a baseline assessment and at 12 weeks after renal neuromodulation for two patients with PCOS.

FIG. 2B is a plot of fasting plasma glucose (mmol/l) at a baseline assessment and at 12 weeks after renal neuromodulation for two patients with PCOS.

FIG. 2C is a plot of insulin sensitivity (mg/kg per min) at a baseline assessment and at 12 weeks after renal neuromodulation for two patients with PCOS.

FIG. 2D is a plot of cystatin C (mg/l) at a baseline assessment and at 12 weeks after renal neuromodulation for two patients with PCOS.

FIG. 2E is a plot of creatinine clearance (ml/min) at a baseline assessment and at 12 weeks after renal neuromodulation for two patients with PCOS.

FIG. 2F is a plot of urinary albumin creatinine ratio (mg/g creatinine) at a baseline assessment and at 12 weeks after renal neuromodulation for two patients with PCOS.

FIG. 3 illustrates an intravascular neuromodulation system configured in accordance with an embodiment of the present technology.

FIG. 4 illustrates modulating renal nerves with a neuromodulation system in accordance with an embodiment of the present technology.

FIG. 5 is a block diagram illustrating a method of modulating renal nerves in accordance with any embodiment of the present technology.

FIG. 6 is a conceptual illustration of the sympathetic nervous system (SNS) and how the brain communicates with the body via the SNS.

FIG. 7 is an enlarged anatomic view of nerves of a left kidney to form the renal plexus surrounding the left renal artery.

FIGS. 8A and 8B are anatomic and conceptual views, respectively, of a human body depicting neural efferent and afferent communication between the brain and kidneys.

FIGS. 9A and 9B are anatomic views of the arterial vasculature and venous vasculature, respectively, of a human.

### DETAILED DESCRIPTION

The present technology is directed to apparatuses, systems, and methods for treating PCOS and related conditions using renal neuromodulation. For example, some embodiments include performing therapeutically-effective renal neuromodulation on a patient diagnosed with PCOS. As discussed in greater detail below, renal neuromodulation can include rendering neural fibers inert, inactive, or otherwise completely or partially reduced in function. This result can be electrically-induced, thermally-induced, or induced by another mechanism during a renal neuromodulation procedure, e.g., a procedure including percutaneous transluminal intravascular access.

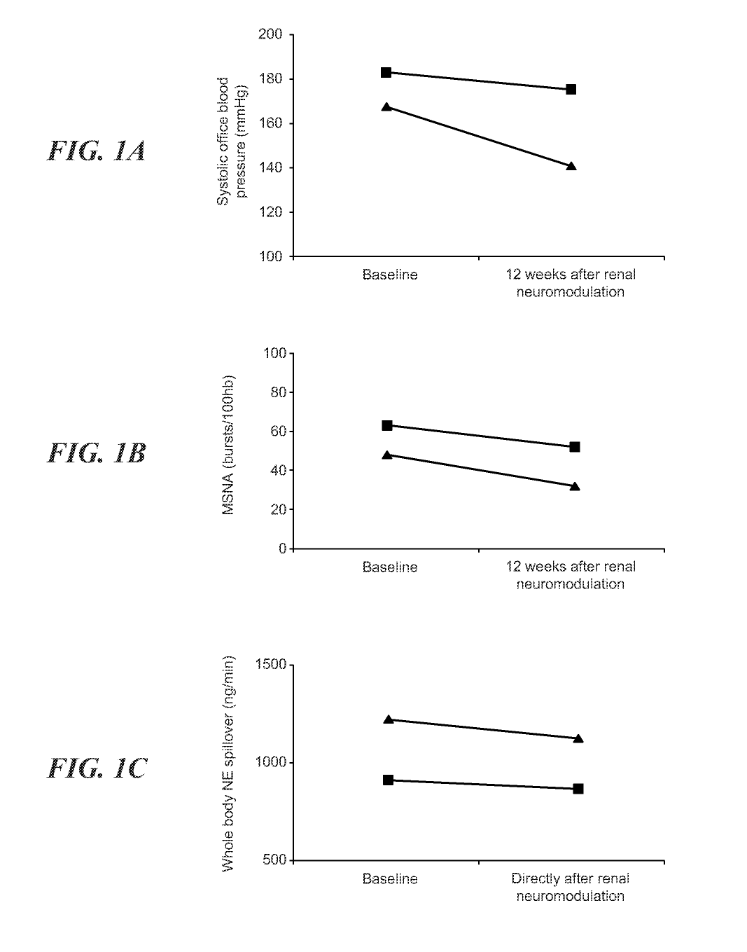

Specific details of several embodiments of the technology are described below with reference to FIGS. 1-9B. Although many of the embodiments are described below with respect to devices, systems, and methods for intravascular modulation of renal nerves using cryotherapeutic and electrode-based approaches, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements and that the technology can have other embodiments without several of the features shown and described below with reference to FIGS. 1-9B.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

### I. POLYCYSTIC OVARY SYNDROME

PCOS is a common endocrine abnormality in women and can be characterized by androgen excess, hyperinsulinemia, and/or other physiological conditions. The etiology of PCOS is uncertain; however evidence suggests that it results from both genetic susceptibility as well as environmental influences including the presence of obesity. The clinical presentation of PCOS can include reproductive (e.g., oligo/amenorrhea, infertility, and hirsutism), metabolic (e.g., obesity, metabolic syndrome, insulin resistance, increased cardiovascular risk profile), and psychological features. PCOS can be characterized by both localized increase in ovarian sympathetic nerve activity (Lara et al., 1993, Endocrinology 133: 2690-2695; incorporated herein by reference in its entirety) and global increase in sympathetic nervous system tone such as muscle sympathetic nerve activity (MSNA) (Sverrisdottir et al., 2008, Am J Physiol Endocrinol Metab 294: E576-581; Stener-Victorin et al., 2009, Am J Physiol Regul Integr Comp Physiol 297: R387-395; incorporated herein by reference in their entireties). Additionally, the degree of sympathoexcitation may be related to the degree of PCOS severity.

Renal sympathetic nerves can contribute to cardiovascular, metabolic, and/or other features that characterize PCOS. For example, among other PCOS presentations, obesity and hypertension can be characterized by increased efferent sympathetic drive to the kidneys and increased systemic sympathetic nerve firing modulated by afferent signaling from renal sensory nerves. The role of renal sympathetic nerves as contributors to the pathogenesis of elevated blood pressure, particularly in obese patients, has been demonstrated both experimentally and in humans. Apart from its role in cardiovascular regulation, sympathetic nervous system activation also has metabolic effects resulting in increased lipolysis and increased levels of fatty acids in plasma, increased hepatic gluconeogenesis, and alterations in pancreatic insulin release. Chronic sympathetic activation predisposes to the development of insulin resistance, which is often associated with obesity and hypertension and can be a key feature of PCOS.

A patient suspected of having PCOS can be positively diagnosed if they present with the following criteria: (1) excess androgen activity, (2) oligoovulation/anovulation and/or polycystic ovaries (assessed, for example, by gynecologic ultrasound or pelvic laparoscopy), and (3) other entities are excluded that would cause excess androgen activity. Androgen excess can be tested by measuring total and free testosterone levels. Androstenedione (an androgen precursor) can also be measured as levels are typically elevated in female patients having PCOS. As examples, polycystic ovaries can be substantiated by a finding of twelve or more follicles measuring 2-9 mm in diameter, or by finding increased ovarian volume (>10 cm³). Further tests for imbalances and/or irregularities in patients suspected of having or having been diagnosed with PCOS using the above criteria can include assessing levels of hormones (e.g., estrogen, FSH, LH, 17-ketosteriods), fasting glucose levels, lipid levels, prolactin levels, thyroid function tests, and pregnancy tests. In further embodiments, PCOS patients or patients suspected of having PCOS can be assessed for elevated sympathetic nerve activity, including establishing measurements for markers of elevated sympathetic nerve activity, including for example, MSNA, total body plasma norepinephrine spillover levels, and heart rate variability.

### II. RENAL NEUROMODULATION

Renal neuromodulation is the partial or complete incapacitation or other effective disruption of nerves innervating the kidneys. In particular, renal neuromodulation can include inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Methods and systems for renal neuromodulation for efficaciously treating several clinical conditions characterized by increased overall sympathetic activity, such as PCOS and associated conditions, are described herein.

Intravascular devices that reduce sympathetic nerve activity by applying, for example, RF energy to a target site in the renal artery have recently been shown to reduce blood pressure in patients with treatment-resistant hypertension. The renal sympathetic nerves arise from T10-L₂ and follow the renal artery to the kidney. The sympathetic nerves innervating the kidneys terminate in the blood vessels, the juxtaglomerular apparatus, and the renal tubules. Stimulation of renal efferent nerves results in increased renin release (and subsequent renin-angiotensin-aldosterone system (RAAS) activation) and sodium retention and decreased renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure).

Various techniques can be used to partially or completely incapacitate neural pathways, such as those innervating the kidney. The purposeful application of energy (e.g., electrical energy, thermal energy) to tissue can induce one or more desired thermal heating and/or cooling effects on localized regions of the renal artery and adjacent regions along all or a portion of the renal plexus RP, which lay intimately within or adjacent to the adventitia of the renal artery. Some embodiments of the present technology, for example, include cryotherapeutic renal neuromodulation, which can include cooling tissue at a target site in a manner that modulates neural function. The mechanisms of cryotherapeutic tissue damage include, for example, direct cell injury (e.g., necrosis), vascular injury (e.g., starving the cell from nutrients by damaging supplying blood vessels), and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Several embodiments of the present technology include cooling a structure at or near an inner surface of a renal artery wall such that proximate (e.g., adjacent) tissue is effectively cooled to a depth where sympathetic renal nerves reside. For example, a cooling structure can be cooled to the extent that it causes therapeutically-effective, cryogenic renal-nerve modulation. Sufficiently cooling at least a portion of a sympathetic renal nerve may slow or potentially block conduction of neural signals to produce a prolonged or permanent reduction in renal sympathetic activity.

As an alternative to or in conjunction with cryotherapeutic cooling, other suitable energy delivery techniques, such as electrode-based approaches, can be used for therapeutically-effective renal neuromodulation. For example, an energy delivery element (e.g., electrode) can be configured to deliver electrical and/or thermal energy at a treatment site. Suitable energy modalities can include, for example, radiofrequency (RF) energy (monopolar and/or bipolar), pulsed RF energy, microwave energy, ultrasound energy, high-intensity focused ultrasound (HIFU) energy, laser, optical energy, magnetic, direct heat, or other suitable energy modalities alone or in combination. Moreover, electrodes (or other energy delivery elements) can be used alone or with other electrodes in a multi-electrode array. Examples of suitable multi-electrode devices are described in U.S. patent application Ser. No. 13/281,360, filed Oct. 25, 2011, and incorporated herein by reference in its entirety. Other suitable devices and technologies, such as cryotherapeutic devices are described in U.S. patent application Ser. No. 13/279,330, filed Oct. 23, 2011, and additional thermal devices are described in U.S. patent application Ser. No. 13/279,205, filed Oct. 21, 2011, each of which are incorporated herein by reference in their entireties.

Thermal effects can include both thermal ablation and non-ablative thermal alteration or damage (e.g., via sustained heating and/or resistive heating) to partially or completely disrupt the ability of a nerve to transmit a signal. Desired thermal heating effects, for example, may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for ablative thermal alteration. More specifically, exposure to thermal energy in excess of a body temperature of about 37° C., but below a temperature of about 45° C., may induce thermal alteration via moderate heating of target neural fibers or of vascular structures that perfuse the target fibers. In cases where vascular structures are affected, the target neural fibers may be denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures that perfuse the target fibers. In some patients, it may be desirable to achieve temperatures that thermally ablate the target neural fibers or the vascular structures, but that are less than about 90° C., or less than about 85° C., or less than about 80° C., and/or less than about 75° C.

### III. METHODS FOR TREATMENT OF POLYCYSTIC OVARY SYNDROME

Disclosed herein are several embodiments of methods directed to treatment of PCOS and related conditions using renal neuromodulation. The methods disclosed herein may represent a significant improvement over conventional approaches and techniques in that they allow for the potential targeting elevated sympathetic drive, which may either be a cause of PCOS or a key mediator of the multiple manifestations of the disease. Also, the disclosed methods provide for localized treatment and limited duration (e.g., one-time treatment) treatment regimes.

In certain embodiments, the methods provided herein comprise performing thermal ablation, thereby decreasing sympathetic renal nerve activity. In certain embodiments, thermal ablation may be repeated one or more times at various intervals until a desired sympathetic nerve activity level or another therapeutic benchmark is reached. In one embodiment, a decrease in sympathetic nerve activity may be observed via a marker of sympathetic nerve activity in PCOS patients, such as decreased levels of plasma norepinephrine (noradrenaline). Other measures or markers of sympathetic nerve activity can include MSNA, sympathetic spillover, urinary or blood markers of renal function, measures of blood or urinary renin or aldosterone, and/or heart rate variability. In another embodiment, other measurable physiological parameters or markers, such as a reduction in androgen production (e.g., lower testosterone levels) and associated symptoms (e.g., acne, hirsutism), increased regularity of menstruation, ovulation, decrease in number of ovarian cysts, reduction in pain level perceived by the PCOS patient, improved blood pressure control, improved blood glucose regulation, etc., can be used to assess efficacy of the thermal ablation treatment for PCOS patients.

In certain embodiments of the methods provided herein, thermal ablation results in a decrease in sympathetic nerve activity over a specific timeframe. In certain of these embodiments, sympathetic nerve activity levels are decreased over an extended timeframe, e.g., within 1 month, 2 months, 3 months, 6, months, 9 months or 12 months post-ablation.

In certain embodiments, the methods disclosed herein may comprise an additional step of measuring sympathetic nerve activity levels, and in certain of these embodiments the methods further comprise comparing the activity level to a baseline activity level. Such comparisons can be used to monitor therapeutic efficacy and to determine when and if to repeat the ablation procedure. In certain embodiments, a baseline sympathetic nerve activity level is derived from the subject undergoing treatment. For example, baseline sympathetic nerve activity level may be measured in the subject at one or more timepoints prior to thermal ablation. A baseline sympathetic nerve activity value may represent sympathetic nerve activity at a specific timepoint before thermal ablation, or it may represent an average activity level at two or more timepoints prior to thermal ablation. In certain embodiments, the baseline value is based on sympathetic nerve activity immediately prior to thermal ablation (e.g., after the subject has already been catheterized). Alternatively, a baseline value may be derived from a standard value for sympathetic nerve activity observed across the population as a whole or across a particular subpopulation. In certain embodiments, post-ablation sympathetic nerve activity levels are measured in extended timeframes post-ablation, e.g., 3 months, 6 months or 12 months post ablation.

In certain embodiments of the methods provided herein, the methods are designed to decrease sympathetic nerve activity to a target level. In these embodiments, the methods include a step of measuring sympathetic nerve activity levels post-ablation (e.g., 6 months post-treatment, 12 months post-treatment, etc.) and comparing the resultant activity level to a baseline activity level as discussed above. In certain of these embodiments, the treatment is repeated until the target sympathetic nerve activity level is reached. In other embodiments, the methods are simply designed to decrease sympathetic nerve activity below a baseline level without requiring a particular target activity level.

Renal neuromodulation may be performed on a patient diagnosed with PCOS to reduce one or more measurable physiological parameters corresponding to the PCOS. In some embodiments, renal neuromodulation may prevent increase, maintain, or reduce the number of ovarian cysts (e.g., immature ovarian follicles). A reduction in the number of ovarian cysts can be, for example, at least about 5%, 10%, or a greater amount as determined by qualitative or quantitative analysis (e.g., ultrasound) before and after (e.g., 1, 3, 6, or 12 months after) a renal neuromodulation procedure. In other embodiments, renal neuromodulation may prevent expansion of, maintain, or reduce an ovarian cyst size with regard to a particular ovarian cyst or an average size of some or all ovarian cysts in a patient. A reduction in ovarian cyst size can be, for example, at least about 5%, 10%, or a greater amount as determined by qualitative or quantitative analysis (e.g., ultrasound) before and after (e.g., 1, 3, 6, or 12 months after) a renal neuromodulation procedure. In other embodiments, abnormally large ovarian size (>10 cm³) may be normalized (or brought closer to a normal range).

In addition to or instead of affecting the growth or size of one or more cysts in a patient, renal neuromodulation may efficaciously treat another measurable physiological parameter or sequela corresponding to PCOS. For example, in some embodiments, renal neuromodulation may reduce the severity and/or frequency of pain, reproductive/fertility issues (e.g., oligo/amenorrhea, infertility, acne and hirsutism), metabolic issues (e.g., obesity, metabolic syndrome, insulin resistance), and cardiovascular risk (e.g., high cholesterol, hypertension). These and other results can occur at various times, e.g., directly following renal neuromodulation or within about one month, three months, six months, a year, or a longer period following renal neuromodulation.

The progression of PCOS may be related to sympathetic overactivity and, correspondingly, the degree of sympathoexcitation in a patient may be related to the severity of the clinical presentation of the PCOS. The kidneys are strategically positioned to be both a cause (via afferent nerve fibers) and a target (via efferent sympathetic nerves) of elevated central sympathetic drive. In some embodiments, renal neuromodulation can be used to reduce central sympathetic drive in a patient diagnosed with PCOS in a manner that treats the patient for the PCOS. For example, muscle sympathetic nerve activity can be reduced by at least about 10% in the patient within about three months after at least partially inhibiting sympathetic neural activity in nerves proximate a renal artery of the kidney. Similarly, whole body norepinephrine spillover can be reduced at least about 20% in the patient within about three months after at least partially inhibiting sympathetic neural activity in nerves proximate a renal artery of the kidney.

In one prophetic example, a patient diagnosed with PCOS can be subjected to a baseline assessment indicating a first set of measurable parameters corresponding to the PCOS. Such parameters can include, for example, blood pressure, cholesterol levels, blood glucose levels, fasting blood insulin levels, measures of insulin sensitivity, measures of blood or urinary renin or aldosterone, blood or urine measures of renal function, duration/frequency of menses, testosterone levels, FSH/LH (luteinizing hormone levels, perceived pain level, severity of hirsutism, and severity of acne. The patient also can be tested (e.g., using ultrasound) to determine a baseline size and number of cysts of the ovaries. Following baseline assessment, the patient can be subjected to a renal neuromodulation procedure. Such a procedure can, for example, include any of the treatment modalities described herein or another treatment modality in accordance with the present technology. The treatment can be performed on nerves proximate one or both kidneys of the patient. Following the treatment (e.g., 1, 3, 6, or 12 months following the treatment), the patient can be subjected to a follow-up assessment. The follow-up assessment can indicate a measurable improvement in one or more physiological parameters corresponding to the PCOS.

The methods described herein address the sympathetic excess that is thought to be an underlying cause of PCOS or a central mechanism through which PCOS manifests its multiple deleterious effects on patients. In contrast, known therapies currently prescribed for PCOS patients typically address only specific manifestations of PCOS. Additionally, conventional therapies require the patient to remain compliant with the treatment regimen over time. In contrast, renal neuromodulation can be a one-time treatment that would be expected to have durable benefits to inhibit the long-term disease progression and thereby achieve a favorable patient outcome.

In one embodiment, patients diagnosed with PCOS can be treated with combinations of therapies for treating both primary causative modes of PCOS as well as sequelae of PCOS. For example, combinations of therapies can be tailored based on specific manifestations of the disease in a particular patient. In a specific example, patients having PCOS and presenting hypertension can be treated with both anti-hypertensive therapy (e.g., drugs) and renal neuromodulation. In another example, renal neuromodulation can be combined with cholesterol lowering agents (e.g., statins), hormonal therapy (e.g., estrogen-progestin contraceptive), fertility treatments (e.g., clomiphene, dexamethasone, FSH injections, ovarian surgery, in vitro fertilization), antiandrogens (e.g., spironolactone, finasteride, cyproterone acetate, GnRH agonsists), acne-focused antibiotics, anti-acne treatments, hair growth inhibitors (e.g., eflornithine hydrochloride) and depilatories for hirsutism as well as weight loss and lifestyle change recommendations/programs.

Treatment of PCOS or related conditions may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

### IV. TREATMENT EXAMPLES

### Example 1

### Effect of Renal Neuromodulation on PCOS

This section describes an example of the clinical use of renal neuromodulation in the treatment of PCOS. Additional embodiments of the present technology may be practiced with features similar to or different than those described with respect to this example. Among other features of the present technology, this example illustrates that renal neuromodulation may have utility in the treatment of PCOS and related conditions. Although this example describes several results observed approximately three months following renal neuromodulation, these and other results can occur at various times, e.g., directly following renal neuromodulation or within about one month, six months, a year, or a longer period following renal neuromodulation.

Two obese patients with hypertension and PCOS were offered to undergo a renal neuromodulation procedure. PCOS was previously diagnosed in both patients by a combination of clinical and biochemical signs of hyperandrogenism and polycystic ovaries on ultrasound imaging. Secondary forms of hypertension were ruled out. Baseline blood pressure levels, anthropometric and biochemical characteristics as well as antihypertensive medication regimens are summarized in Table 1. Lifestyle and medication were stable for at least four weeks prior to the baseline assessment and the patients did not change their lifestyle and medication during the three months between the renal neuromodulation and the follow-up assessment. Following the baseline assessment of sympathetic nerve activity (using microneurography (MSNA) and norepinephrine spillover measurements) and insulin sensitivity (using euglycemic hyperinsulinemic clamp), both patients underwent bilateral radiofrequency renal neuromodulation without any periprocedural complications. Measurements of cystatin-C, creatinine clearance, and urinary albumin creatinine ratio were also obtained. All measurements performed at the baseline assessment were repeated three months after the renal neuromodulation at the follow-up assessment.

TABLE 1
Clinical and Biochemical Parameters and Antihypertensive
Medication at Baseline and Three Months after Bilateral
Renal Neuromodulation for Each of the Two Patients

| | Patient No. 1 | | Patient No. 2 | |
|---|---|---|---|---|
| | (27 years) | | (34 years) | |
| | BL | 3 M BL | BL | 3 M BL |
| Weight (kg) | 97.6 | 95.1 | 90.4 | 92.8 |
| BMI (kg/m²) | 36.2 | 35.4 | 34.3 | 35.4 |
| SBP (mmHg) | 183 | 175 | 167 | 140 |
| DBP (mmHg) | 107 | 81 | 123 | 102 |
| Heart rate (beats/min) | 89 | 85 | 93 | 77 |
| Sodium (mmol/l) | 140 | 138 | 138 | 140 |
| Potasium (mmol/l) | 3.9 | 4.1 | 3.9 | 4.3 |
| Creatinine (mmol/l) | 64 | 68 | 68 | 69 |
| eGFR (ml/min per 1.73 m²) | >90 | 89 | 86 | 84 |
| Urea (mmol/l) | 4.3 | 4.5 | 6.1 | 6.3 |
| Fasting glucose (mmol/l) | 6.1 | 4.3 | 5.4 | 5 |

Antihypertensive medication (mg/day)

| | | | | |
|---|---|---|---|---|
| Irbesartan/HCT | 300/25 | 300/25 | — | — |
| Methyldopa | 750 | 750 | — | — |
| Prazosin | 10 | 10 | — | — |
| Spironolactone | 25 | 25 | 100 | 100 |
| Amlodipine/valsartan | — | — | 5/160 | 5/160 |
| Ramipril | — | — | 20 | 20 |
| Moxonidine | — | — | 0.4 | 0.4 |

BL, baseline; eGFR, estimated glomerular filtration rate; FU, follow-up; HCT, hydrochlorothiazide; M, month

MSNA was recorded using microneurography in the peroneal nerve. A tracer infusion of 3H-labeled norepinephrine (levo-7-3HNE, specific activity of 11-25 Ci/mmol; New England Nuclear, Boston, Mass., USA) was given via a peripheral vein at 0.6-0.8 μCi/min, after a priming bolus of 11 μCi, for the measurement of total body norepinephrine spillover by isotope dilution. The euglycemic hyperinsulinemic clamp technique was used to quantify in-vivo insulin sensitivity. After a bolus injection of 9 mU/kg insulin (Actrapid HM100 U/ml; Novo Nordisk, Baulkham Hills, New South Wales, Australia), a constant infusion rate of 40 mU/m² per minute was maintained over two hours. Blood glucose concentration was clamped at the euglycemic level of 5 mmol/l through the variable infusion of 25% glucose and measured every 5 minutes using an autoanalyzer (ABL 800 Basic; Radiometer, Copenhagen). Peripheral insulin sensitivity was derived from the average glucose infusion rate during the final 20 minutes, corrected for glucose space, and normalized to body weight.

Both patients had uncontrolled clinic blood pressure levels at baseline despite a therapeutic regimen consisting of at least four different antihypertensive drug classes and had a BMI in the obese range (Table 1, FIG. 1A). Of note, patient one was intolerant to calcium channel blockers and patient two to thiazide diuretics. Neither of the patients was on oral antidiabetic drugs or insulin before or during the study. Both patients had normal renal function as indicated by cystatin-C levels below 1 mg/l. As shown in FIGS. 1B and 1C, indices of sympathetic nervous system activation were substantially elevated in both patients with an approximately 2.5 to 3-fold increase above levels typically found in normotensive healthy controls for both MSNA (normal being about 15 to 20 bursts/min) and whole body norepinephrine (NE) spillover (normal being about 300 to 600 ng/min).

Bilateral renal neuromodulation resulted in mild-to-moderate reductions in SBP and DBP in the two patients at the three-month follow-up (Table 1, FIG. 1A). MSNA was reduced in both patients by about 17% and about 33%, respectively (FIG. 1B) after renal neuromodulation. Whole body NE spillover was well above the upper normal limit of around 600 ng/min in both patients at baseline and reduced in both patients by 5% and 8% directly after renal neuromodulation (FIG. 1C), and by 28% in the one patient who had whole body NE spillover repeated at 12 weeks, suggesting that sympathetic activation may decrease further over time.

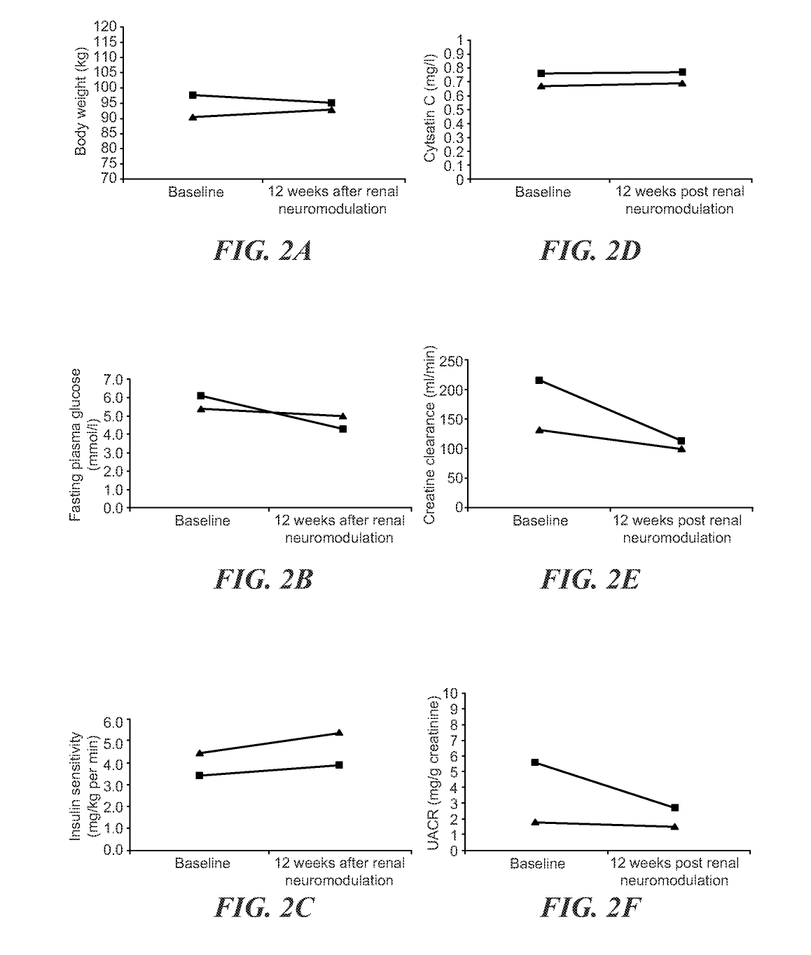

Changes in metabolic parameters following bilateral renal neuromodulation are illustrated in FIGS. 2A-2F. There was no substantial change in body weight with one patient experiencing a minor reduction and the other patient a minor increase in body weight at the three-month follow-up (FIG. 2A). Fasting plasma glucose levels were lower in both patients at the three-month follow-up compared to baseline (FIG. 2B). Insulin sensitivity, as assessed by euglycemic hyperinsulinemic clamp, increased by 20.9% and 14.4%, respectively, in both patients at the three-month follow-up after renal neuromodulation (FIG. 2C). There was no indication of renal function impairment after renal neuromodulation with cystatin-C levels being unchanged or reduced (FIG. 2D). Assessment of creatinine clearance at baseline, though limited in accurately assessing glomerular filtration, revealed a state of hyperfiltration in one of the two patients (216 and 132 ml/min, respectively), which was normalized three months after renal neuromodulation (FIG. 2E). One patient presented with microalbuminuria at baseline, which was substantially reduced by approximately 50% at the three-month follow-up after renal neuromodulation (FIG. 2F).

As discussed above, PCOS has been associated with increased sympathetic nerve activity. The reduction of central sympathetic drive associated with renal neuromodulation may highlight the relevance of sympathetic activation in blood pressure control and glucose metabolism in patients with PCOS. Indeed, sympathetic activation may be a link between obesity, hypertension, and insulin resistance, which are frequently encountered in PCOS and represent an important target for the prevention and treatment of the metabolic and cardiovascular features of PCOS. The findings discussed in this example suggest an inhibitory effect of renal neuromodulation on indices of sympathetic activation that was associated with simultaneous reduction in both blood pressure and insulin resistance. Similar findings on insulin resistance have been reported with pharmaceutical agents that reduce central sympathetic drive, such as moxonidine. However, selectively removing the renal contribution to central sympathetic drive, without causing further systemic pharmacologic interactions, allows a direct examination of the relation between sympathetic drive and insulin resistance.

The findings suggest that reduction of sympathetic activity, as measured by MSNA and norepinephrine spillover, via renal sympathetic neuromodulation resulted in improved fasting glucose levels and insulin sensitivity in the absence of significant changes in body weight and any alterations in lifestyle or antihypertensive medication. A likely explanation for the substantial improvement in insulin sensitivity in response to renal neuromodulation is a combination of beneficial effects of sympathoinhibition and reduced release of norepinephrine on regional hemodynamics and direct cellular effects.

In the human forearm, increased norepinephrine release typically results in a substantial reduction in forearm blood flow and typically is associated with a markedly reduced forearm uptake of glucose. This can highlight the adverse effect of sympathetic activation on the ability of the cell to transport glucose across its membrane, a hallmark of insulin resistance. This can be the result of a reduced number of open capillaries due to vasoconstriction and/or an increase in the distance that insulin must travel to reach the cell membrane from the intravascular compartment. Furthermore, this situation can be perpetuated if insulin resistance already exists, which can reduce the ability of insulin to increase muscle perfusion (e.g., by approximately 30%). The relevance of these hemodynamic consequences of sympathetic activation is highlighted by studies demonstrating a direct relationship between the sympathetic nerve firing rate to skeletal muscle tissue and insulin resistance and an inverse relationship between insulin resistance and the number of open capillaries.

In addition to the beneficial hemodynamic and metabolic effects, it is also relevant that renal sympathetic neuromodulation likely does not impair renal function and may perhaps be renoprotective in patients with insulin resistance and glomerular hyperfiltration. Although cystatin-C levels were essentially unchanged after the renal neuromodulation procedure, it is of interest that glomerular hyperfiltration, as reflected by a creatinine clearance above 150 ml/min and evident in one patient, was normalized after the procedure. Glomerular hyperfiltration is considered to be a progression factor for renal impairment and reversing hyperfiltration may result in improved renal and cardiovascular outcomes.

Although hormone levels were not measured, it is striking that one of the two patients who was amenorrheic for the previous 3 years resumed irregular menses approximately 6 weeks after the renal neuromodulation procedure. In this context, it is important to note that increased sympathetic inputs to the ovary have been linked to the etiology of PCOS in an experimental model of PCOS. In the same model, ablation of the sympathetic nerves extending to endocrine cells of the ovary restored a normal steroidal response and resulted in initiation of estrous cyclicity and ovulation. In line with these experimental findings, human polycystic ovaries are characterized by increased catecholaminergic nerve density and bilateral wedge resection, performed after failure of standard hormonal therapy to partially denervate the ovaries, has been shown to recover normal ovarian function.

The findings discussed in this example suggest that a localized, single intervention specifically targeting the renal nerves may beneficially influence several important aspects of PCOS. By way of theory, targeting both afferent and efferent renal nerves via a catheter-based approach may cause beneficial effects extending well beyond the kidneys and systemic blood pressure. The role of sympathetic activation for blood pressure regulation is well established, as is the relevance of increased renal sympathetic nerve activity for the alterations in renal blood flow and glomerular filtration rate. There is now also clear evidence that sympathetic activation results in adverse consequences on metabolic control, including insulin sensitivity. Additionally, overactivity of the sympathetic nervous system is implicated in the etiology of PCOS. Methods of treating PCOS patients using renal neuromodulation are derived from the recognition described herein that the kidneys are strategically positioned to be both a cause (via afferent nerve fibers) and a target (via efferent sympathetic nerves) of elevated central sympathetic drive, as mirrored by the substantial increase in MSNA in both PCOS patients.

Components of PCOS contributing to elevated cardiovascular risk, such as elevated blood pressure, insulin sensitivity, glomerular hyperfiltration, and microalbuminuria were all influenced beneficially by the renal neuromodulation procedure discussed in this example without any adverse side effects. Accordingly, renal neuromodulation can provide a tool to interfere with the fundamental processes underpinning the etiology of PCOS.

In some embodiments, renal neuromodulation may prevent expansion of and/or reduce the size of ovarian cysts in a patient, such as a patent diagnosed with PCOS. Without being bound by theory, it is believed that the sympathetic nervous system may impact fluid retention in ovarian cysts and that renal neuromodulation may treat this inappropriate fluid retention. Although the size of ovarian cysts was not quantified in this example, such a result could be quantified, e.g., using ultrasound. In a hypothetical example, renal neuromodulation may prevent expansion of, maintain, or reduce an ovarian-cyst size with regard to a particular ovarian cyst or an average size of some or all ovarian cysts in a patient. A reduction in ovarian-cyst size can be, for example, at least about 5%, 10%, or a greater amount as determined by qualitative or quantitative analysis (e.g., ultrasound) before and after (e.g., 1, 3, 6, or 12 months after) a renal neuromodulation procedure. In another example, renal neuromodulation may reduce a number of ovarian cysts and/or prevent additional ovarian cysts from forming in a patient. A reduction in a number of ovarian cysts can be, for example, at least about 5%, 10%, or a greater amount as determined by qualitative or quantitative analysis (e.g., ultrasound) before and after (e.g., 1, 3, 6, or 12 months after) a renal neuromodulation procedure. Reduction in ovarian cyst size or number could also be assessed by noting that an abnormally large ovarian size (>10 cm³) may be normalized (or brought closer to a normal range).

### Example 2

### Effect of Renal Neuromodulation on Hypertension

Patients were selected having a baseline systolic blood pressure of 160 mm Hg or more (≧150 mm Hg for patients with type 2 diabetes) and taking three or more antihypertensive drugs, and were randomly allocated into two groups: 51 assessed in a control group (antihypertensive drugs only) and 49 assessed in a treated group (undergone renal neuromodulation and antihypertensive drugs).

Patients in both groups were assessed at 6 months. Office-based blood pressure measurements in the treated group were reduced by 32/12 mm Hg (SD 23/11, baseline of 178/96 mm Hg, p<0.0001), whereas they did not differ from baseline in the control group (change of 1/0 mm Hg, baseline of 178/97 mm Hg, p=0.77 systolic and p=0.83 diastolic). Between-group differences in blood pressure at 6 months were 33/11 mm Hg (p<0.0001). At 6 months, 41 (84%) of 49 patients who underwent renal neuromodulation had a reduction in systolic blood pressure of 10 mm Hg or more, compared with 18 (35%) of 51 control patients (p<0.0001).

### V. SELECTED EMBODIMENTS OF RENAL NEUROMODULATION SYSTEMS, DEVICES AND METHODS

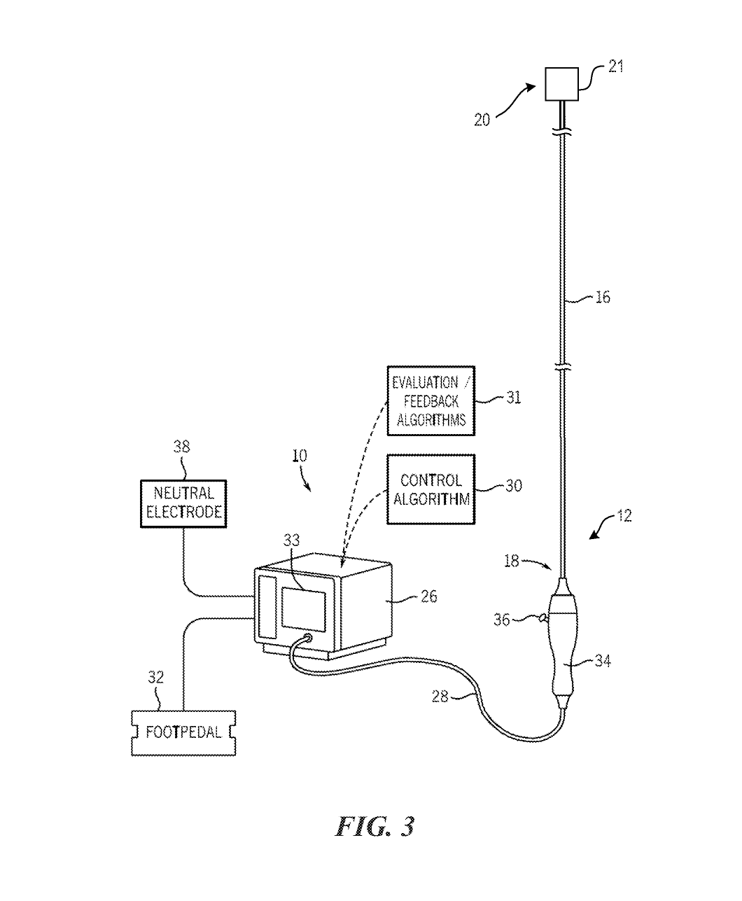

FIG. 3 illustrates a renal neuromodulation system 10 configured in accordance with an embodiment of the present technology. The system 10, for example, may be used to perform therapeutically-effective renal neuromodulation on a patient diagnosed with PCOS. The system 10 includes an intravascular treatment device 12 operably coupled to an energy source or console 26 (e.g., a radiofrequency energy generator, a cryotherapy console). In the embodiment shown in FIG. 3, the treatment device 12 (e.g., a catheter) includes an elongated shaft 16 having a proximal portion 18, a handle 34 at a proximal region of the proximal portion 18, and a distal portion 20 extending distally relative to the proximal portion 18. The treatment device 12 further includes a neuromodulation assembly or treatment section 21 at the distal portion 20 of the shaft 16. The neuromodulation assembly 21 can include one or more electrodes or energy-delivery elements, a cryotherapeutic cooling assembly and/or a nerve monitoring device configured to be delivered to a renal blood vessel (e.g., a renal artery) in a low-profile configuration.

Upon delivery to a target treatment site within a renal blood vessel, the neuromodulation assembly 21 can be further configured to be deployed into a treatment state or arrangement for delivering energy at the treatment site and providing therapeutically-effective electrically-induced and/or thermally-induced renal neuromodulation. In some embodiments, the neuromodulation assembly 21 may be placed or transformed into the deployed state or arrangement via remote actuation, e.g., via an actuator 36, such as a knob, pin, or lever carried by the handle 34. In other embodiments, however, the neuromodulation assembly 21 may be transformed between the delivery and deployed states using other suitable mechanisms or techniques. The proximal end of the neuromodulation assembly 21 can be carried by or affixed to the distal portion 20 of the elongated shaft 16. A distal end of the neuromodulation assembly 21 may terminate with, for example, an atraumatic rounded tip or cap. Alternatively, the distal end of the neuromodulation assembly 21 may be configured to engage another element of the system 10 or treatment device 12. For example, the distal end of the neuromodulation assembly 21 may define a passageway for engaging a guide wire (not shown) for delivery of the treatment device using over-the-wire (“OTW”) or rapid exchange (“RX”) techniques.

The energy source or console 26 can be configured to generate a selected form and magnitude of energy for delivery to the target treatment site via the neuromodulation assembly 21. A control mechanism, such as a foot pedal 32, may be connected (e.g., pneumatically connected or electrically connected) to the energy source or console 26 to allow an operator to initiate, terminate and, optionally, adjust various operational characteristics of the energy source or console 26, including, but not limited to, power delivery. The system 10 may also include a remote control device (not shown) that can be positioned in a sterile filed and operably coupled to the neuromodulation assembly 21. The remote control device can be configured to allow for selective activation of the neuromodulation assembly 21. In other embodiments, the remote control device may be built into the handle assembly 34. The energy source 26 can be configured to deliver the treatment energy via an automated control algorithm 30 and/or under the control of the clinician. In addition, the energy source 26 may include one or more evaluation or feedback algorithms 31 to provide feedback to the clinician before, during, and/or after therapy.

The energy source 26 can further include a device or monitor that may include processing circuitry, such as a microprocessor, and a display 33. The processing circuitry may be configured to execute stored instructions relating to the control algorithm 30. The energy source 26 may be configured to communicate with the treatment device 12 (e.g., via a cable 28) to control the neuromodulation assembly and/or to send signals to or receive signals from the nerve monitoring device. The display 33 may be configured to provide indications of power levels or sensor data, such as audio, visual or other indications, or may be configured to communicate information to another device. For example, the console 26 may also be configured to be operably coupled to a catheter lab screen or system for displaying treatment information, such as nerve activity before and/or after treatment.

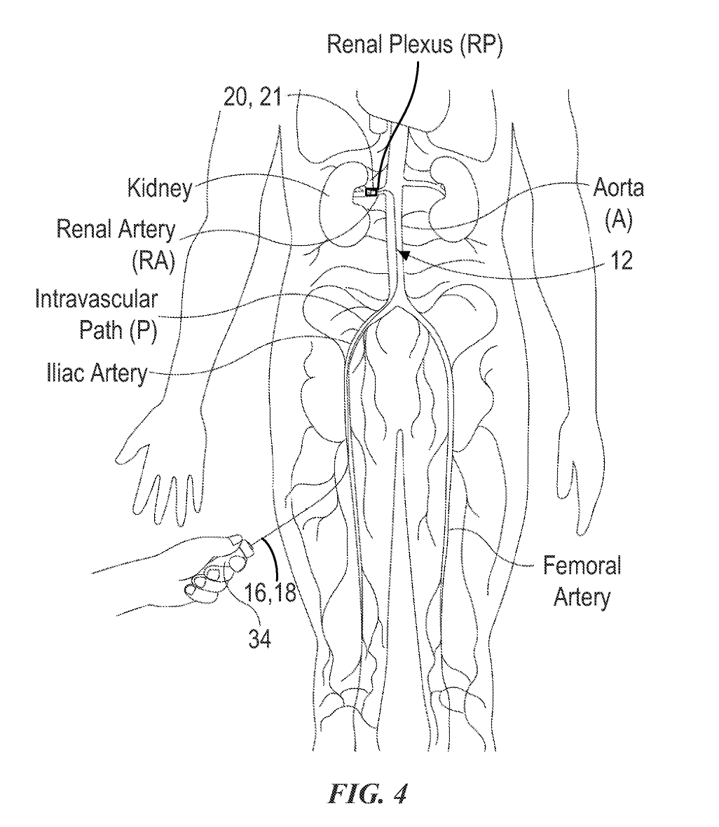

FIG. 4 illustrates modulating renal nerves with an embodiment of the system 10. The treatment device 12 provides access to the renal plexus RP through an intravascular path P, such as a percutaneous access site in the femoral (illustrated), brachial, radial, or axillary artery to a targeted treatment site within a respective renal artery RA. As illustrated, a section of the proximal portion 18 of the shaft 16 is exposed externally of the patient. By manipulating the proximal portion 18 of the shaft 16 from outside the intravascular path P, the clinician may advance the shaft 16 through the sometimes tortuous intravascular path P and remotely manipulate the distal portion 20 of the shaft 16. Image guidance, e.g., computed tomography (CT), fluoroscopy, intravascular ultrasound (IVUS), optical coherence tomography (OCT), or another suitable guidance modality, or combinations thereof, may be used to aid the clinician's manipulation. Further, in some embodiments, image guidance components (e.g., IVUS, OCT) may be incorporated into the treatment device 12.

After the neuromodulation assembly 21 is adequately positioned in the renal artery RA, it can be radially expanded or otherwise deployed using the handle 34 or other suitable control mechanism until the neuromodulation assembly is positioned at its target site and in stable contact with the inner wall of the renal artery RA. The purposeful application of energy from the neuromodulation assembly can then be applied to tissue to induce one or more desired neuromodulating effects on localized regions of the renal artery RA and adjacent regions of the renal plexus RP, which lay intimately within, adjacent to, or in close proximity to the adventitia of the renal artery RA. The neuromodulating effects may include denervation, thermal ablation, and non-ablative thermal alteration or damage (e.g., via sustained heating and/or resistive heating). The purposeful application of the energy may achieve neuromodulation along all or at least a portion of the renal plexus RP.

As mentioned previously, the methods disclosed herein may use a variety of suitable energy modalities, including RF energy, microwave energy, laser, optical energy, ultrasound, HIFU, magnetic energy, direct heat, cryotherapy, or a combination thereof. Alternatively or in addition to these techniques, the methods may utilize one or more non-ablative neuromodulatory techniques. For example, the methods may utilize non-ablative SNS denervation by removal of target nerves, injection of target nerves with a destructive drug or pharmaceutical compound, or treatment of the target nerves with non-ablative energy modalities. In certain embodiments, the amount of reduction of the sympathetic nerve activity may vary depending on the specific technique being used.

In certain embodiments, a neuromodulation device for use in the methods disclosed herein may combine two or more energy modalities. For example, the device may include both a hyperthermic source of ablative energy and a hypothermic source, making it capable of, for example, performing both RF ablation and cryoablation. The distal end of the treatment device may be straight (for example, a focal catheter), expandable (for example, an expanding mesh or cryoballoon), or have any other configuration (e.g., a helical coil as shown in FIG. 16 and FIG. 17). Additionally or alternatively, the treatment device may be configured to carry out one or more non-ablative neuromodulatory techniques. For example, the device may comprise a means for diffusing a drug or pharmaceutical compound at the target treatment area (e.g., a distal spray nozzle).

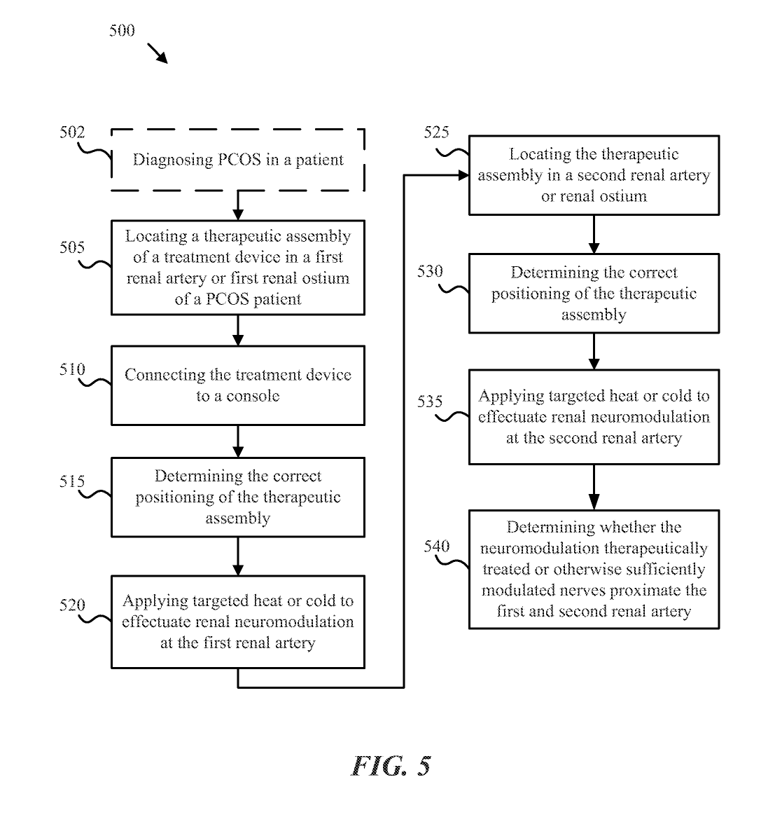

FIG. 5 is a block diagram illustrating a method 500 of modulating renal nerves using the system 10 described above with reference to FIGS. 3 and 4. With reference to FIGS. 3-5 together, the method 500 can optionally include diagnosing PCOS in a patient (if not yet determined) and/or selecting a suitable candidate PCOS patient for performing renal neuromodulation (block 502). The method 500 can include intravascularly locating the neuromodulation assembly 21 in a delivery state (e.g., low-profile configuration) to a first target site in or near a first renal blood vessel (e.g., first renal artery) or first renal ostium (block 505). The treatment device 12 and/or portions thereof (e.g., the neuromodulation assembly 21) can be inserted into a guide catheter or sheath to facilitate intravascular delivery of the neuromodulation assembly 21. In certain embodiments, for example, the treatment device 12 can be configured to fit within an 8 Fr guide catheter or smaller (e.g., 7 Fr, 6 Fr, etc.) to access small peripheral vessels. A guide wire (not shown) can be used to manipulate and enhance control of the shaft 16 and the neuromodulation assembly 21 (e.g., in an over-the-wire or a rapid-exchange configuration). In some embodiments, radiopaque markers and/or markings on the treatment device 12 and/or the guide wire can facilitate placement of the neuromodulation assembly 21 at the first target site (e.g., a first renal artery or first renal ostium of a PCOS patient). In some embodiments, a contrast material can be delivered distally beyond the neuromodulation assembly 21, and fluoroscopy and/or other suitable imaging techniques can be used to aid in placement of the neuromodulation assembly 21 at the first target site.

The method 500 can further include connecting the treatment device 12 to the console 26 (block 510), and determining whether the neuromodulation assembly 21 is in the correct position at the target site and/or whether the neuromodulation assembly electrodes (or cryotherapy balloon) is functioning properly (block 515). Once the neuromodulation assembly 21 is properly located at the first target site and no malfunctions are detected, the console 26 can be manipulated to initiate application of an energy field to the target site to cause electrically-induced and/or thermally-induced partial or full denervation of the kidney (e.g., using electrodes or cryotherapeutic devices). Accordingly, heating and/or cooling of the neuromodulation assembly 21 causes modulation of renal nerves at the first target site to cause partial or full denervation of the kidney associated with the first target site (block 520).

In a specific example, the treatment device 12 can be a cryogenic device and cryogenic cooling can be applied for one or more cycles (e.g., for 30 second increments, 60 second increments, 90 second increments, etc.) in one or more locations along the circumference and/or length of the first renal artery or first renal ostium. The cooling cycles can be, for example, fixed periods or can be fully or partially dependent on detected temperatures (e.g., temperatures detected by a thermocouple (not shown) of the cooling assembly 130). In some embodiments, a first stage can include cooling tissue until a first target temperature is reached. A second stage can include maintaining cooling for a set period, such as 15-180 seconds (e.g., 90 seconds). A third stage can include terminating or decreasing cooling to allow the tissue to warm to a second target temperature higher than the first target temperature. A fourth stage can include continuing to allow the tissue to warm for a set period, such as 10-120 seconds (e.g., 60 seconds). A fifth stage can include cooling the tissue until the first target temperature (or a different target temperature) is reached. A sixth stage can include maintaining cooling for a set period, such as 15-180 seconds (e.g., 90 seconds). A seventh stage can, for example, include allowing the tissue to warm completely (e.g., to reach a body temperature).

The neuromodulation assembly 21 can then be located at a second target site in or near a second renal blood vessel (e.g., second renal artery) or second renal ostium (block 525), and correct positioning of the assembly 21 can be determined (block 530). In selected embodiments, a contrast material can be delivered distally beyond the neuromodulation assembly 21 and fluoroscopy and/or other suitable imaging techniques can be used to locate the second renal artery. The method 500 continues by applying targeted heat or cold to effectuate renal neuromodulation at the second target site to cause partial or full denervation of the kidney associated with the second target site (block 535).

After providing the therapeutically-effective neuromodulation energy (e.g., cryogenic cooling, RF energy, ultrasound energy, etc.), the method 500 may also include determining whether the neuromodulation therapeutically treated the patient for PCOS or otherwise sufficiently modulated nerves or other neural structures proximate the first and second target sites (block 540). For example, the process of determining whether the neuromodulation therapeutically treated the nerves can include determining whether nerves were sufficiently denervated or otherwise disrupted to reduce, suppress, inhibit, block or otherwise affect the afferent and/or efferent renal signals. In a further embodiment, PCOS patient assessment could be performed at time intervals (e.g., 1 month, 3 months, 6 months, 12 months) following neuromodulation treatment. For example, the PCOS patient can be assessed for measurements of perceived pain, blood pressure control, blood glucose levels, androgen levels (e.g., testosterone levels), imaging-based measurements of ovarian cyst size and number, markers of renal injury (e.g., serum BUN levels, serum creatinine levels, serum cystatin C levels, proteinuria levels, and NGAL and Kim-1 levels), and measures of sympathetic activity (e.g., MSNA, renal and/or total body spillover, plasma norepinephrine levels, and heart rate variability).

In other embodiments, various steps in the method 500 can be modified, omitted, and/or additional steps may be added. In further embodiments, the method 500 can have a delay between applying therapeutically-effective neuromodulation energy to a first target site at or near a first renal artery or first renal ostium and applying therapeutically-effective neuromodulation energy to a second target site at or near a second renal artery or second renal ostium. For example, neuromodulation of the first renal artery can take place at a first treatment session, and neuromodulation of the second renal artery can take place a second treatment session at a later time.

### VI. PERTINENT ANATOMY AND PHYSIOLOGY

The following discussion provides further details regarding pertinent patient anatomy and physiology. This section is intended to supplement and expand upon the previous discussion regarding the relevant anatomy and physiology, and to provide additional context regarding the disclosed technology and the therapeutic benefits associated with renal neuromodulation. For example, as mentioned previously, several properties of the renal vasculature may inform the design of treatment devices and associated methods for achieving renal neuromodulation via intravascular access, and impose specific design requirements for such devices. Specific design requirements may include accessing the renal artery, facilitating stable contact between the energy delivery elements of such devices and a luminal surface or wall of the renal artery, and/or effectively modulating the renal nerves with the neuromodulatory apparatus.

A. The Sympathetic Nervous System

The SNS is a branch of the autonomic nervous system along with the enteric nervous system and parasympathetic nervous system. It is always active at a basal level (called sympathetic tone) and becomes more active during times of stress. Like other parts of the nervous system, the SNS operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine binds adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The SNS is responsible for up- and down-regulation of many homeostatic mechanisms in living organisms. Fibers from the SNS innervate tissues in almost every organ system, providing at least some regulatory function to physiological features as diverse as pupil diameter, gut motility, and urinary output. This response is also known as the sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the SNS and indirectly via catecholamines secreted from the adrenal medulla.

Science typically looks at the SNS as an automatic regulation system, that is, one that operates without the intervention of conscious thought. Some evolutionary theorists suggest that the SNS operated in early organisms to maintain survival as the SNS is responsible for priming the body for action. One example of this priming is in the moments before waking, in which sympathetic outflow spontaneously increases in preparation for action.

1. The Sympathetic Chain

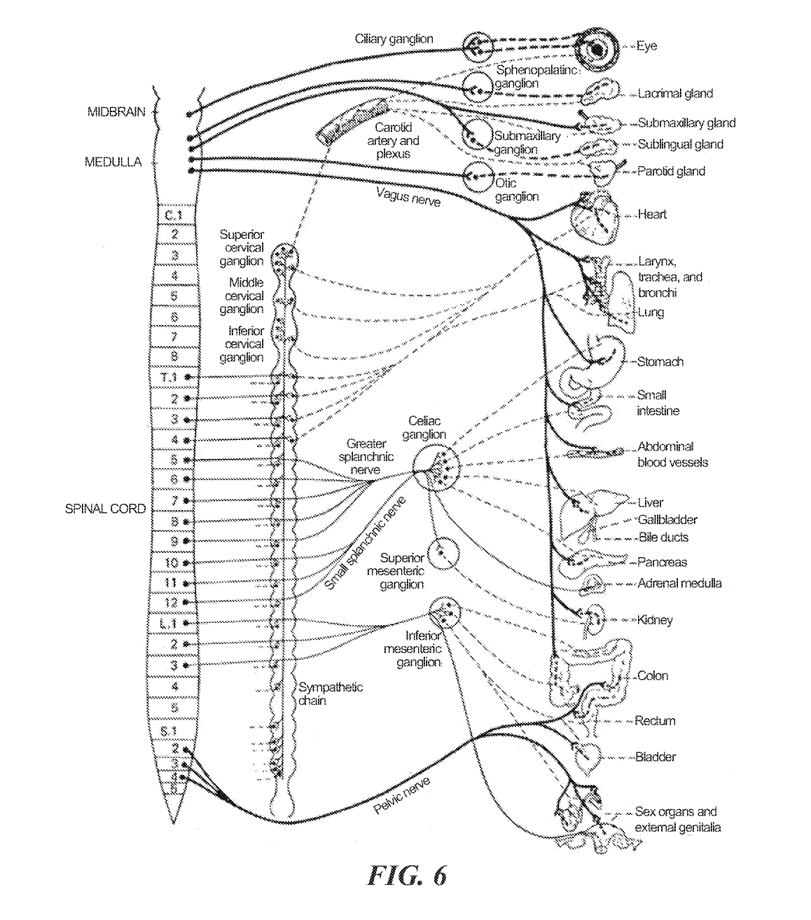

As shown in FIG. 6, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors that connect to either the paravertebral (which lie near the vertebral column) or prevertebral (which lie near the aortic bifurcation) ganglia extending alongside the spinal column.

In order to reach the target organs and glands, the axons travel long distances in the body. Many axons relay their message to a second cell through synaptic transmission. The first cell (the presynaptic cell) sends a neurotransmitter across the synaptic cleft (the space between the axon terminal of the first cell and the dendrite of the second cell) where it activates the second cell (the postsynaptic cell). The message is then propagated to the final destination.

In the SNS and other neuronal networks of the peripheral nervous system, these synapses are located at sites called ganglia, discussed above. The cell that sends its fiber to a ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands. The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

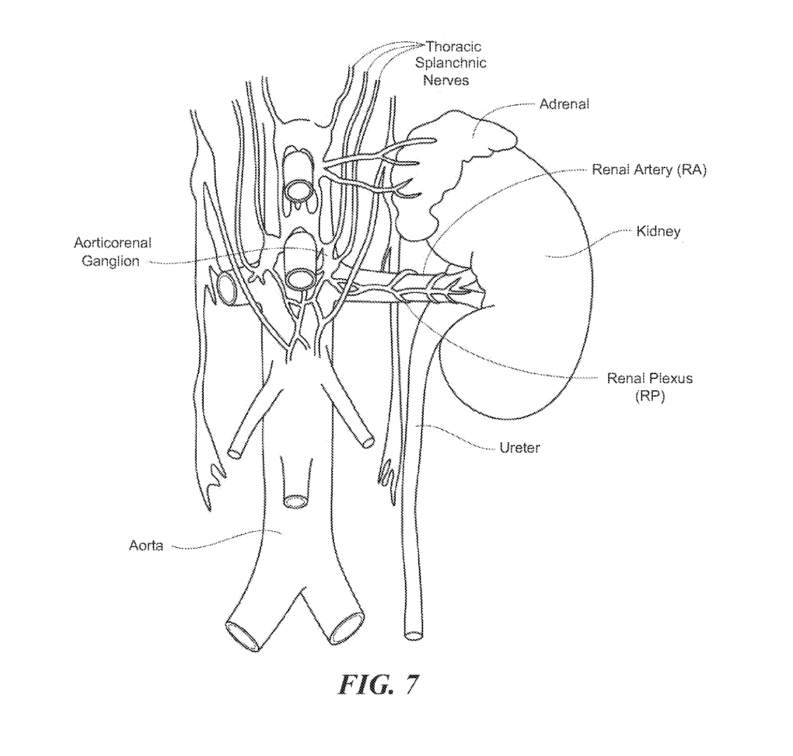

As FIG. 7 shows, the kidney is innervated by the renal plexus RP, which is intimately associated with the renal artery RA. The renal plexus RP is an autonomic plexus that surrounds the renal artery RA and is embedded within the adventitia of the renal artery RA. The renal plexus RP extends along the renal artery RA until it arrives at the substance of the kidney. Fibers contributing to the renal plexus RP arise from the celiac ganglion, the superior mesenteric ganglion, the aorticorenal ganglion and the aortic plexus. The renal plexus RP, also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, and the second lumbar splanchnic nerve, and they travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus RP and are distributed to the renal vasculature.

3. Renal Sympathetic Neural Activity

Messages travel through the SNS in a bidirectional flow. Efferent messages may trigger changes in different parts of the body simultaneously. For example, the SNS may accelerate heart rate; widen bronchial passages; decrease motility (movement) of the large intestine; constrict blood vessels; increase peristalsis in the esophagus; cause pupil dilation, cause piloerection (i.e., goose bumps), cause perspiration (i.e., sweating), and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing overactivity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine (NE) from the kidneys to plasma revealed increased renal NE spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced SNS overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end-stage renal disease are characterized by heightened sympathetic nervous activation. In patients with end-stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well known adverse consequences of chronic sympathetic overactivity, such as hypertension, left ventricular hypertrophy, ventricular arrhythmias, sudden cardiac death, insulin resistance, diabetes, and metabolic syndrome.

(i) Renal Sympathetic Efferent Nerve Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

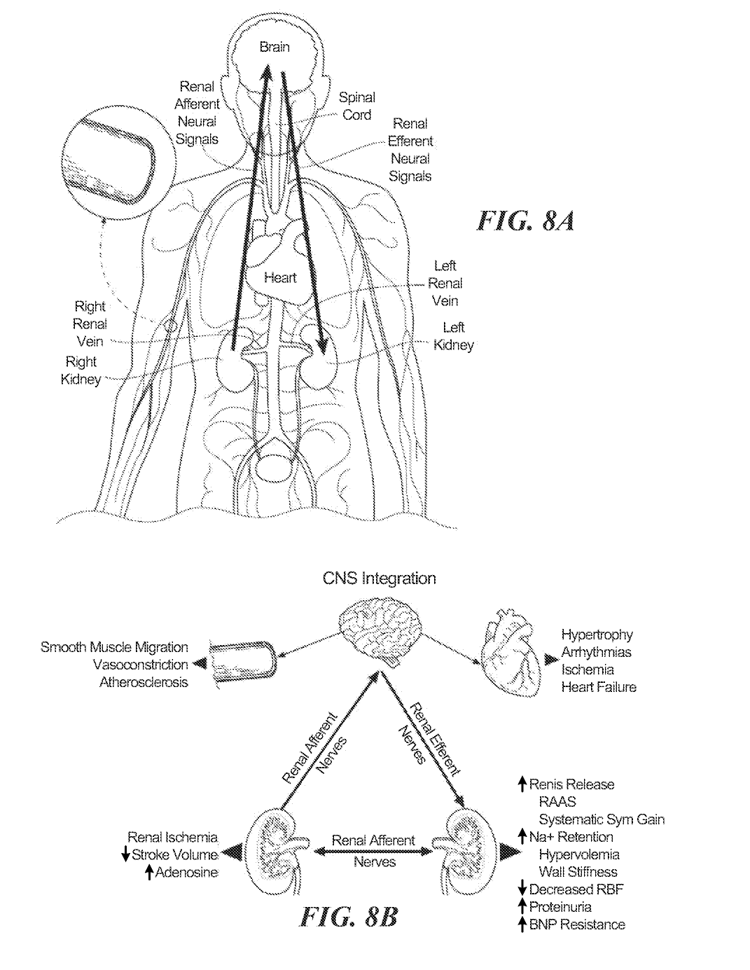

The kidneys communicate with integral structures in the CNS via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 8B and 8B, this afferent communication might be from the kidney to the brain or might be from one kidney to the other kidney (via the CNS). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic overactivity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and renal blood flow, and (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Neuromodulation

As provided above, renal neuromodulation is likely to be valuable in the treatment of several clinical conditions characterized by increased overall and particularly renal sympathetic activity such as hypertension, metabolic syndrome, insulin resistance, diabetes, left ventricular hypertrophy, chronic end-stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 6. For example, as previously discussed, a reduction in central sympathetic drive may reduce the insulin resistance that afflicts people with metabolic syndrome and Type II diabetes. Additionally, patients with osteoporosis are also sympathetically activated and might also benefit from the down regulation of sympathetic drive that accompanies renal denervation.

C. Achieving Intravascular Access to the Renal Artery

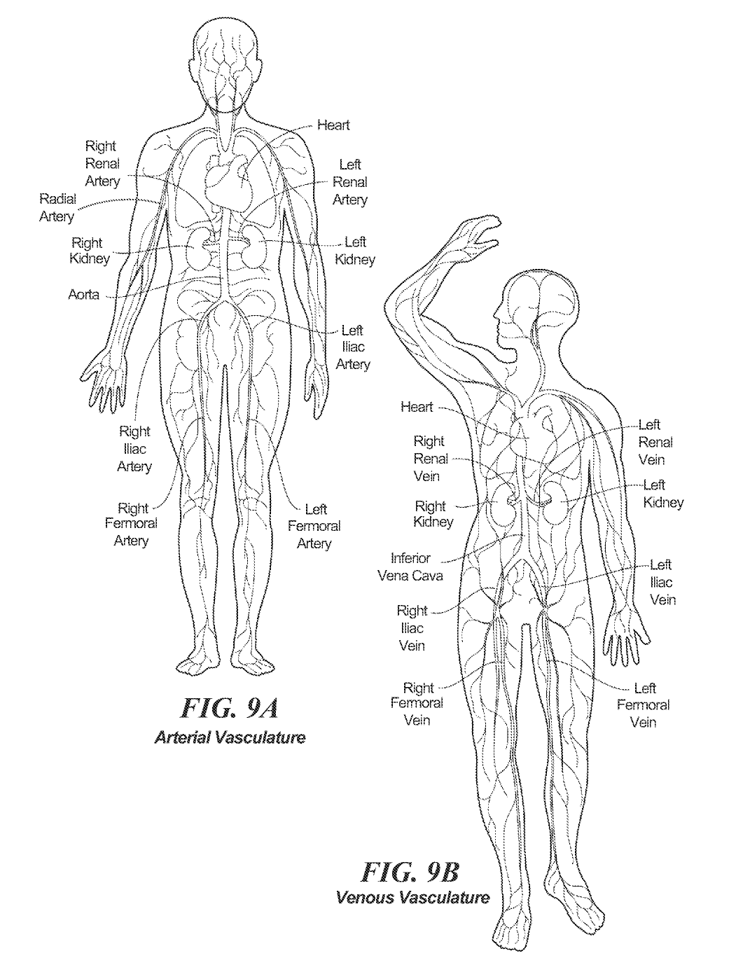

In accordance with the present technology, neuromodulation of a left and/or right renal plexus RP, which is intimately associated with a left and/or right renal artery, may be achieved through intravascular access. As FIG. 9A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 9B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus RP may be achieved in accordance with embodiments of the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as polycystic kidney disease, hypertension, other chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access can account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, or a cryotherapeutic device, consistent positioning and appropriate contact force applied by the energy or cryotherapy delivery element to the vessel wall, and adhesion between the applicator and the vessel wall can be important for predictability. However, navigation can be impeded by the tight space within a renal artery RA, as well as tortuosity of the artery. Furthermore, establishing consistent contact can be complicated by patient movement, respiration, and/or the cardiac cycle because these factors may cause significant movement of the renal artery RA relative to the aorta, and the cardiac cycle may transiently distend the renal artery RA (i.e., cause the wall of the artery to pulse).

After accessing a renal artery and facilitating stable contact between neuromodulatory apparatus and a luminal surface of the artery, nerves in and around the adventitia of the artery can be modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy can be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery RA can be applied carefully. Accordingly, the complex fluid mechanics and thermodynamic conditions present in the renal artery during treatment, particularly those that may impact heat transfer dynamics at the treatment site, may be important in applying energy (e.g., hearting thermal energy) and/or removing heat from the tissue (e.g., cooling thermal conditions) from within the renal artery.

The neuromodulatory apparatus can also be configured to allow for adjustable positioning and repositioning of an energy delivery element or a cryotherapeutic device, within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery via the cryotherapeutic devices or energy delivery elements and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

Blood flow through a renal artery may be temporarily occluded for a short time with minimal or no complications. However, occlusion for a significant amount of time can be avoided in some cases to prevent injury to the kidney such as ischemia. It can be beneficial to avoid occlusion altogether or, if occlusion is beneficial, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the takeoff angle of a renal artery relative to the aorta. These properties will be discussed in greater detail with respect to the renal arteries. However, depending on the apparatus, systems, and methods utilized to achieve renal neuromodulation, such properties of the renal arteries also may guide and/or constrain design characteristics.

As noted above, an apparatus positioned within a renal artery can conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite intima-media thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment can be important to reach the target neural fibers, the treatment typically is not too deep (e.g., the treatment can be less than about 5 mm from inner wall of the renal artery) so as to avoid non-target tissue and anatomical structures such as the renal vein.

An additional property of the renal artery that may be of interest is the degree of renal motion relative to the aorta, induced by respiration and/or blood flow pulsatility. A patient's kidney, which is located at the distal end of the renal artery, may move as much as four inches cranially with respiratory excursion. This may impart significant motion to the renal artery connecting the aorta and the kidney. Accordingly, the neuromodulatory apparatus can have a unique balance of stiffness and flexibility to maintain contact between a cryo-applicator or another thermal treatment element and the vessel wall during cycles of respiration. Furthermore, the takeoff angle between the renal artery and the aorta may vary significantly between patients, and also may vary dynamically within a patient, e.g., due to kidney motion. The takeoff angle generally may be in a range of about 30°-135°.

### VII. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. For example, in additional embodiments, the system 10 may include a treatment device configured to deliver therapeutic energy to the patient from an external location outside the patient's body, i.e., without direct or close contact to the target site. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of treating a human patient diagnosed with polycystic ovary syndrome, comprising:
intravascularly positioning a neuromodulation assembly within a renal blood vessel of the patient and adjacent to a renal nerve of the patient;
at least partially inhibiting sympathetic neural activity in nerves proximate a renal artery of a kidney of the patient; and
reducing central sympathetic drive in the patient in a manner that treats the patient for the polycystic ovary syndrome.

2. The method of claim 1 wherein reducing central sympathetic drive in the patient in a manner that treats the patient for the polycystic ovary syndrome includes reducing expansion of, maintaining the size of, or reducing the size of an ovarian cyst in the patient.

3. The method of claim 1 wherein reducing central sympathetic drive in the patient in a manner that treats the patient for the polycystic ovary syndrome includes reducing the size of an ovarian cyst in the patient at least about 5% within about three months to about 12 months after at least partially inhibiting sympathetic neural activity in nerves proximate the renal artery of the kidney of the patient.

4. The method of claim 1 wherein reducing central sympathetic drive in the patient in a manner that treats the patient for the polycystic ovary syndrome includes reducing a number of ovarian cysts in the patient at least about 5% within about three months to about 12 months after at least partially inhibiting sympathetic neural activity in nerves proximate the renal artery of the kidney of the patient.

5. The method of claim 1 wherein reducing central sympathetic drive in the patient in a manner that treats the patient for the polycystic ovary syndrome includes reducing muscle sympathetic nerve activity in the patient.

6. The method of claim 1 wherein reducing central sympathetic drive in the patient in a manner that treats the patient for the polycystic ovary syndrome includes reducing whole body norepinephrine spillover in the patient.

7. The method of claim 1 wherein reducing central sympathetic drive in the patient in a manner that treats the patient for the polycystic ovary syndrome includes increasing insulin sensitivity in the patient.

8. The method of claim 1 wherein the polycystic ovary syndrome is associated with a condition including oligo/amenorrhea and reducing central sympathetic drive in the patient in a manner that treats the patient for the polycystic ovary syndrome includes causing resumption of menses in the patient within about three months to about 12 months after at least partially inhibiting sympathetic neural activity in nerves proximate the renal artery of the kidney of the patient.

9. The method of claim 1 wherein at least partially inhibiting sympathetic neural activity in nerves proximate the renal artery of the kidney of the patient includes at least partially inhibiting afferent neural activity.

10. The method of claim 1 wherein at least partially inhibiting sympathetic neural activity in nerves proximate the renal artery of the kidney of the patient includes at least partially inhibiting efferent neural activity.

11. The method of claim 1 wherein at least partially inhibiting sympathetic neural activity in nerves proximate the renal artery of the kidney of the patient includes modulating a renal nerve of the patient via an intravascularly positioned catheter carrying a neuromodulation assembly positioned at least proximate to the renal nerve.

12. The method of claim 11 wherein modulating the renal nerve includes thermally modulating the renal nerve from within the renal artery of the patient.

13. The method of claim 12 wherein thermally modulating the renal nerve includes cryotherapeutically cooling the renal nerve.

14. The method of claim 12 wherein thermally modulating the renal nerve includes delivering an energy field to the renal nerve.

15. A method, comprising:
percutaneously introducing a neuromodulation assembly at a distal portion of a treatment device proximate to neural fibers innervating a kidney of a human patient diagnosed with polycystic ovary syndrome;
partially disrupting function of the neural fibers by applying thermal energy to the neural fibers via the neuromodulation assembly; and
removing the neuromodulation assembly from the patient after treatment;
wherein partial disruption of the function of the neural fibers therapeutically treats the diagnosed polycystic kidney disease.

16. The method of claim 15, further comprising improving one or more physiological parameters corresponding to the polycystic ovary syndrome.

17. The method of claim 16 wherein improving one or more physiological parameters corresponding to the polycystic ovary syndrome includes reducing at least one of androgen levels, blood glucose levels, blood pressure, acne and hirsutism.

18. A method for treating polycystic ovary syndrome in a human patient comprising:
positioning an energy delivery element of a renal denervation catheter within a renal blood vessel of the patient and adjacent to post-ganglionic neural fibers that innervate a kidney of the patient; and
at least partially ablating a renal nerve of the patient via the energy delivery element;
wherein at least partially ablating the renal nerve results in a therapeutically beneficial reduction in one or more physiological conditions associated with polycystic ovary syndrome of the patient.

19. The method of claim 18, further comprising administering one or more pharmaceutical drugs to the patient, wherein the pharmaceutical drugs are selected from the group consisting of antihypertensive drugs, hormone therapy drugs and anti-diabetic drugs.

20. The method of claim 18 wherein the reduction in one or more physiological conditions associated with polycystic ovary syndrome includes a reduction in the number of ovarian cysts in the patient.
